(19) United States
(12) Patent Application Publication
(10) Pub. No.: US 2005/0175672 A1
(43) Pub. Date: Aug. 11, 2005

(54) Particulate plant sterol compositions

(76) Inventors: Michael D. Kluetz, Watertown, MN (US); Robert L. Klein, Wayzata, MN (US); Stephen K. Snyder, Plymouth, MN (US); Melanie J. Goulson, Dayton, MN (US); Vincent M. Cavallini, Edina, MN (US)

Correspondence Address: Edward Levine, Esq.;Cargill, Incorporated (Nutraceuticals), 15407 McGinty Road West, Wayzata, MN, 55391, US

(21) Appl. No.: 10/775,933

(22) Filed: Feb. 10, 2004

(51) Int. Cl. A61K 47/00; A23L 2/00

(52) U.S. Cl. 424/439; 426/590; 426/601

(57) ABSTRACT

Particulate plant sterol compositions having defined particle size distribution (PSD) characteristics are provided. Processes for preparing the compositions and methods for dispersing the compositions in aqueous media are also provided.

### TECHNICAL FIELD

This invention relates to plant sterols, and more particularly to particulate plant sterol compositions having defined particle size distribution (PSD) characteristics, methods for making the same, and food and beverage compositions incorporating the same.

### BACKGROUND

Coronary heart disease (CHD) is a common and serious form of cardiovascular disease that causes a significant number of deaths in the U.S. each year. Research has shown that plant sterols, including phytosterols, phytostanols, and esters of the same, can lower total and LDL cholesterol and reduce the risk of CHD. For example, the FDA has authorized the labeling of foods as useful for reducing the risk of CHD when supplemented with plant sterols. Because plant sterols are very hydrophobic compounds, they typically have been incorporated into fat-based foods such as margarines or salad dressings. In other food applications, plant sterols have been mixed with emulsifiers in order to achieve water dispersibility, although often at emulsifier concentrations that can introduce off-flavors and that can significantly dilute the concentration of plant sterols.

### SUMMARY

The invention provides particulate plant sterol compositions and methods for making the same. Particulate plant sterol compositions of the invention are useful for dispersion in aqueous media, including aqueous food and beverage products. When dispersed in aqueous media, such as a juice, the compositions do not impart gritty, chalky, or otherundesirable sensory qualities (i.e. with respect to color, flavor, and mouth feel) to the aqueous media. Methods for preparing the compositions are also provided, including one-pass milling methods that avoid the need for size classification and recycling of undesirably sized particles.

In one aspect, the invention provides a composition comprising one or more particulate plant sterols. The composition demonstrates a multi-peak volume-weighted or mass-weighted particle size distribution (PSD) and a multi-peak surface-area-weighted PSD of the one or more particulate plant sterols. The composition, when dispersed in a test juice, has an acceptable mouthfeel in the test juice.

A multi-peak volume- or mass-weighted PSD can demonstrate a first peak of particulate plant sterols having a diameter less than 2 microns, with a volume-weighted mean particle diameter of about 0.3 to about 0.5 microns; and a second peak of particulate plant sterols having a diameter in the range from 2 to about 35 microns, with a volume-weighted mean particle diameter of about 8 to about 12 microns. A second peak can represent from about 65% to about 85% of the volume- or mass-weighted PSD, and the first peak can represent from about 15% to about 35% of the volume- or mass-weighted PSD. In another aspect, the volume-percentage of all particulate plant sterols having a diameter greater than 35 microns in a volume- or mass-weighted PSD can be less than about 3%, or less than about 0.5%.

A composition provided herein can demonstrate a multi-peak surface area-weighted PSD of the one or more particulate plant sterols. A surface area-weighted PSD can demonstrate a first peak of particulate plant sterols having a diameter less than 2 microns; and a second peak of particulate plant sterols having a diameter in the range from 2 to about 35 microns, where the second peak has a surface-area-weighted mean particle diameter of about 8 to 12 microns. The first peak of particulate plant sterols can represent from about 78% to about 92% of the surface-area weighted PSD. The first peak of particulate plant sterols having a diameter less than 2 microns can have a surface-area weighted mean particle diameter of about 0.5 microns or less. The first peak of particulate plant sterols having a diameter less than 2 microns can have a surface-area weighted mean particle diameter of from about 0.3 microns to about 0.5 microns, or about 0.4 microns.

In another aspect, a composition can have a total specific surface area of a multi-peak surface area-weighted PSD of greater than about 2 m²/g, from about 2.5 to about 7 m²/g, or from about 2.8 to about 6.5 m²/g.

In another embodiment, the invention provides compositions including particulate plant sterols that are dispersible or have been dispersed in an aqueous medium. For example, a composition can be an aqueous composition. In other cases, a composition is a powdered composition. A composition can be a food or beverage composition. A beverage composition can be selected from the group consisting of a juice, a juice concentrate, coffee, tea, a smoothie, a shake, soy milk, rice milk, a frappe, a milk fluid, a meal replacement beverage, a diet beverage, and a nutritional supplement beverage. A food composition can be selected from the group consisting of a bread, a baked good, candy, ice cream, a confection, an egg, an egg replacement, ice cream, yogurt, a health supplement, a meal replacement food, and a nutritional supplement.

A composition that includes a dispersion of one or more particulate plant sterols in an aqueous material can demonstrate no or only a slight detectable chalky mouthfeel. A particulate plant sterol composition can be mixed or dispersed in an aqueous material in order to substantially avoid an undesirable sensory attribute in an aqueous dispersion of particulate plant sterols. An undesirable sensory attribute can be a chalky, gritty, drying, or powdery mouthfeel.

In another aspect, the invention provides a process for preparing a particulate plant sterol composition. The process includes cooling a plant sterol starting material; and subjecting the cooled plant sterol starting material to impact or attrition milling. A plant sterol starting material may not include an emulsifier. About 88%-100% (e.g., about 88, 89, 90, 91, 92, 93, 94, 95, 96, 97, 98, 99, or 100% ) by weight of a plant sterol starting material can be 1 or more plant sterols. A plant sterol starting material can include vitamin E and/or tocopherol. A plant sterol starting material can be in the form of pastilles having a diameter of from about 1 mm to about 4 mm, e.g., about 2 mm.

A plant sterol starting material can be cooled in the range from about −100° F. to about −275° F., or from about −175 to about −250° F., or to about −225° F. A plant sterol starting material can be cooled with liquid nitrogen. When cooled with liquid nitrogen, milling of a plant sterol starting material can be performed in an inert (e.g., N₂ gas) atmosphere.

Impact or attrition milling can be performed with a gap mill. A gap mill can have a rotor-stator gap in the range of from about 0.025″ to about 0.05″, or about 0.03″. Impact or attrition milling can be performed in a single pass. A gap mill can have an average tip speed of from about 110 m/s to about 150 m/s, or from about 120 to about 135 m/s. A particulate plant sterol composition can be discharged from a gap mill at a temperature from about −25 to about −275° F., or at a temperature from about −40 to about −75° F., or at about −40 to about −50° F. Impact or attrition milling can be performed in an inert atmosphere. A cooled plant sterol starting material can be subjected to impact or attrition milling in the presence of one or more of the following: a flow agent, a colorant, a flavorant, a vitamin, a mineral, a source of fiber, a protein, and a nutritional additive.

In another embodiment, the invention provides a process for preparing a particulate plant sterol composition that includes milling a plant sterol starting material in a vortex mill having an inlet air pressure of from about 5 to about 6 bar and an outlet temperature of less than about 100° F. Milling can be performed at a temperature from about 60 to about 80° F. and can be performed in a single pass and/or in an inert (e.g., N₂ gas) atmosphere. A plant sterol starting material can be as described previously. A plant sterol starting material can be milled in the presence of one or more of the following: a flow agent, a colorant, a flavorant, a vitamin, a mineral, a source of fiber, a protein, and a nutritional additive.

In another embodiment, the invention provides a method for preparing an aqueous dispersion of a particulate plant sterol composition. The method includes mixing a particulate plant sterol composition with an aqueous material, where the particulate plant sterol composition demonstrates a multi-peak surface area-weighted PSD, as described above.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION

The invention provides compositions having one or more plant sterols in the form of particles having particular PSD characteristics. The particulate plant sterol compositions can be dispersed in aqueous media, such as certain food and beverage products, without imparting a gritty or chalky mouthfeel to the product. Methods for preparing particulate plant sterol compositions, including one-pass methods, are also described.

Plant Sterol Compounds

Particulate compositions provided herein can contain one or more plant sterol compounds. The term “plant sterol” includes, without limitation, phytosterols, phytosterol esters, phytostanols, and phytostanol esters. Phytosterols (and phytosterol esters) are typically naturally occurring substances present in the diet as minor components of vegetable oils, while phytostanols (and phytostanol esters) are hydrogenation compounds of the phytosterols.

Plant sterols for use herein can include any of various positional isomer and stereoisomeric forms, such as α-, β-, or γ-isomers. Typical phytosterol compounds include α-sitosterol, γ-sitosterol, β-sitosterol, campesterol, stigmasterol, brassicasterol, spinosterol, taraxasterol, desmosterol, chalinosterol, poriferasterol, clionasterol, ergosterol, Δ-5-avenosterol, Δ-5-campesterol, clerosterol, Δ-5-stigmasterol, Δ-7,25-stigmadienol, Δ-7-avenosterol, Δ-7-β-sitosterol, and Δ-7-brassicasterol.

Suitable examples of phytosterol esters include, without limitation, β-sitosterol laurate ester, α-sitosterol laurate ester, γ-sitosterol laurate ester, campesterol myristearate ester, stigmasterol oleate ester, campesterol stearate ester, β-sitosterol oleate ester, β-sitosterol palmitate ester, β-sitosterol linoleate ester, α-sitosterol oleate ester, γ-sitosterol oleate ester, β-sitosterol myristearate ester, β-sitosterol ricinoleate ester, campesterol laurate ester, campesterol ricinoleate ester, campesterol oleate ester, campesterol linoleate ester, stigmasterol linoleate ester, stigmasterol laurate ester, stigmasterol caproate ester, α-sitosterol stearate ester, γ-sitosterol stearate ester, α-sitosterol myristearate ester, γ-sitosterol palmitate ester, campesterol ricinoleate ester, stigmasterol ricinoleate ester, campesterol ricinoleate ester, and stigmasterol stearate ester.

Useful phytostanol compounds include α-, β-, and γ-sitostanol, campestanol, stigmastanol, spinostanol, taraxastanol, brassicastanol, desmostanol, chalinostanol, poriferastanol, clionastanol, and ergostanol.

Finally, phytostanol esters for inclusion in a composition provided herein include, without limitation, β-sitostanol laurate ester, campestanol myristearate ester, stigmastanol oleate ester, campestanol stearate ester, β-sitostanol oleate ester, β-sitostanol palmitate ester, β-sitostanol linoleate ester, β-sitostanol myristearate ester, β-sitostanol ricinoleate ester, campestanol laurate ester, campestanol ricinoleate ester, campestanol oleate ester, campestanol linoleate ester, stigmastanol linoleate ester, stigmastanol laurate ester, stigmastanol caproate ester, stigmastanol stearate ester, α-sitostanol laurate ester, γ-sitostanol laurate ester, α-sitostanol oleate ester, γ-sitostanol oleate ester, α-sitostanol stearate ester, γ-sitostanol stearate ester, α-sitostanol myristearate ester, γ-sitostanol palmitate ester, campestanol ricinoleate ester, stigmastanol ricinoleate ester, campestanol ricinoleate ester, β-sitostanol, α-sitostanol, γ-sitostanol, campestanol, and stigmastanol.

Plant Sterol Starting Materials

Typically, a particulate plant sterol composition is prepared from a plant sterol starting material, e.g., as described in the methods below. Plant sterol starting materials can include one or more plant sterol compounds, as described above. For example, a plant sterol starting material can include multiple plant sterol compounds (e.g., 2, 3, 4, 5, 6, 7, 8, 9, 10, 11, 12, 13, 14, 15, 20 or more) in any relative ratio (e.g., 1:1, 2:1, 3:1, 4:1, 5:1, 6:1, 7:1, 8:1, 9:1,or 10:1).

Plant sterol starting materials can be derived from a variety of plant sources, e.g., rice bran oil, corn fiber oil, corn germ oil, wheat germ oil, safflower oil, oat oil, olive oil, cotton seed oil, soybean oil, peanut oil, canola oil, tea, sesame seed oil, grapeseed oil, rapeseed oil, linseed oil, tall oil and other oils obtained from wood pulp, and various other brassica crops. Although plant sterols are typically derived from plants, a plant sterol can also be synthetically prepared, e.g., it need not be derived from a plant source. Additionally, plant sterol starting materials can be prepared as mixtures of individual purified or synthesized plant sterol compounds or can be co-products resulting from purifications of other products (e.g., from plant sources). For example, a plant sterol starting material can be obtained as a co-product of the manufacture of vitamin E and/or tocopherols from vegetable oil deodorizer distillate.

Depending on the application, plant sterol starting materials may or may not contain additional ingredients. For example, certain plant sterol starting materials can contain vitamin E and/or one or more tocopherols, e.g., when the starting material is obtained as a co-product of the manufacture of vitamin E. In some cases, about 88%-100% (e.g.,88, 89, 90, 91, 92, 93, 94, 95, 96, 97, 98, or 99%) of a plant sterol starting material is made up of 1 or more plant sterol compounds. For example, in one embodiment, a plant sterol starting material is made up of about 85-90% (e.g., about 85, 86, 87, 88, 89, or 90%) by weight a mixture of β-sitosterol, campesterol, and stigmasterol. In other cases, a plant sterol starting material can be made up of about 88%-100% (e.g., about 88, 89, 90, 91, 92, 93, 94, 95, 96, 97, 98, 99, or 100%) by weight a mixture of β-sitosterol, campesterol, stigmasterol, brassicasterol, campestanol, β-sitostanol, and Δ-5-avenosterol. In certain cases, a plant sterol starting material can be about 89%-91% by weight a mixture of β-sitosterol, campesterol, stigmasterol, brassicasterol, campestanol, β-sitostanol, and Δ-5-avenosterol.

In certain applications, a phytosterol starting material does not include an emulsifier (e.g., lecithin, mono- and/or di-glycerides, soribitan esters, sucrose esters). While not being bound by theory, it is believed that the addition of these ingredients can contribute to unwanted sensory attributes (e.g., with respect to color, flavor, or mouthfeel) of the compositions.

In yet other applications, one or more of the following is included in a plant sterol starting material: a flow agent (e.g., sodium aluminosilicate, potassium ferrocyanide); a colorant (e.g., beta-carotene); a vitamin or mineral (e.g., vitamins A, C, D, E, and K, and the B vitamins; and the minerals Ca, Fe, Mg, Zn, K, and Se); fiber (both soluble and insoluble; e.g., barley β-fiber, oat β-fiber, mannans, galactomannas, wheat, oat, corn and barley brans); a protein (e.g., amino acids, soy proteins, milk or egg proteins); or a nutritional additive (e.g., gingko biloba, ginseng, chondroitin, glucosamine, echinacea, chromium picolinate, folic acid, soy isoflavones, citrus flavonoids, saw palmetto, sterol glycosides, and flavolignans).

Plant sterol starting materials can be in any form, e.g., pastilles, waxy crude solids, or powders. For example, plant sterol starting materials can be obtained by crystallization of an impure tocopherol-sterol mixture, which is dried, melted, and formed into pastilles about 1 to about 4 mm (e.g., 1, 2, 3, or 4 mm) in diameter. Plant sterol compounds and plant sterol starting materials (e.g., sterol pastilles) can be obtained commercially from, e.g., Cargill, Incorporated (Minneapolis, Minn.), Loders and Croklaan (Channahon, Ill.), Cognis Nutrition and Health (La Grange, Ill.), Forbes Meditech (Vancouver, B.C. Canada), and ADM (Decatur, Ill.). In addition, plant sterol compounds and starting materials can be synthesized and/or obtained from plant sources (e.g., as described in U.S. Pat. Nos. 6,411,206; 5,502,045; 6,087,353; and 4,897,224).

Particle Size Distribution Characteristics of Particulate Plant Sterol Compositions

Particulate plant sterol compositions provided herein can be described by their PSD characteristics. PSD characteristics can be measured with a particle-size analyzer that measures both Mie-scattered and Fraunhofer-diffracted light, e.g., the Horiba Model LA-910 Particle Size Analyzer. Typically, particulate plant sterol compositions demonstrate particular volume- or mass-weighted PSD characteristics and particular surface-area-weighted PSD characteristics. Without being bound by theory, it is believed that particulate plant sterol compositions that demonstrate the described PSD characteristics do not impart chalky, gritty, powdery, oily, or other undesirable sensory attributes (i.e., with respect to mouthfeel, flavor, and color) to compositions (e.g., food, beverage, or aqueous compositions) in which they are dispersed.

For example, a plant sterol composition can demonstrate a multi-peak volume- or mass-weighted particle size distribution (PSD) of the one or more particulate plant sterols. As used herein, the term “multi-peak” means that a PSD demonstrates a distribution possessing at least two distinct modes or peak maxima. In certain cases, a multi-peak PSD can be bimodal.

For example, a volume- or mass-weighted PSD can demonstrate a first peak of particulate plant sterols having a diameter less than 2 microns, and a volume-weighted mean particle diameter of about 5 microns or less (e.g., about 3 to about 5 microns, or about 0.3, 0.35, 0.4, 0.45, or 0.5 microns). As used herein, use of the terms “first,” “second,” “third,” etc. to describe PSD peaks does not imply a time-dependence of appearance or measurement of such peaks nor does it reflect on the magnitude of such peaks. A PSD peak of particulate plant sterols having a diameter less than 2 microns can be referred to herein as a “fines” peak. A first peak in the volume- or mass-weighted PSD can represent from about 15% to about 35% of the total volume- or mass-weighted PSD, or any value therebetween (e.g., about 16, 17, 18, 19, 20, 21, 22, 23, 24, 25, 26, 27, 28, 29, 30, 31, 32, 33, or 34%). A particulate plant sterol composition can demonstrate a volume-or mass-weighted multi-peak PSD having a second peak of particulate plant sterols having a diameter in the range from 2 to about 35 microns. The second peak can have a volume-weighted mean particle diameter of about 8 to about 12 microns, or any value therebetween (e.g., about 8.5, 9, 9.5, 10, 10.5, 11, or 11.5 microns). The second peak can represent from about 65% to about 85% of the total volume- or mass-weighted PSD, or any value therebetween (e.g., about 66, 67, 68, 69, 70, 71, 72, 73, 74, 75, 76, 77, 78, 79, 80, 81, 82, 83, or 84%). In addition, the volume-percentage contributed by all particulate plant sterols having a diameter greater than 35 microns in the volume- or mass-weighted PSD is less than about 3% of the total volume- or mass-weighted PSD, e.g., less than about 2.5%, 2%, 1.5%, 1% or 0.5%.

Particulate plant sterol compositions described herein also demonstrate a multi-peak surface-area-weighted PSD. A multi-peak surface-area-weighted PSD can be bimodal. A surface-area-weighted PSD can demonstrate a first peak of particulate plant sterols having a diameter less than 2 microns, e.g., a “fines” peak. A first peak of particulate plant sterols having a diameter less than 2 microns (e.g., a “fines” peak) can have a surface-area weighted mean particle diameter of from about 0.3 to about 0.5 microns, e.g., about 0.3, 0.35, 0.4, 0.45, or 0.5 microns. A first peak of particulate plant sterols can represent from about 78% to about 92% of the surface-area weighted PSD. A multi-peak surface-area-weighted PSD can demonstrate a second peak of particulate plant sterols having a diameter in the range from 2 to about 35 microns, with a surface-area weighted mean particle diameter of about 8 to 12 microns (e.g., about 8.5, 9, 9.5, 10, 10.5, 11, or 11.5 microns).

The total specific surface area of a PSD can also be a useful parameter for evaluating particulate plant sterol compositions. In the compositions of the present invention, the total specific surface area of a multi-peak surface area-weighted PSD can be greater than about 2 m²/g, e.g., ranging from about 2.5 to about 7 m²/g, or from about 2.8 to about 6.5 m²/g.

Other Physical Characteristics ofParticulate Plant Sterol Compositions

Particulate plant sterol compositions can be in a variety of forms, e.g., solid or liquid. For example, a particulate plant sterol composition can be in powdered form, e.g., immediately after preparation or for subsequent dispersion in an aqueous medium. Alternatively, a particulate plant sterol composition can be an aqueous composition having particulate plant sterols dispersed therein. An aqueous composition can be a food or beverage composition that contains water. For example, particulate plant sterol compositions can be dispersed in beverages such as a juice (e.g., a fruit juice such as orange, grape, cranberry, apple, kiwi, mango, peach, pineapple, plum, cherry, banana, guava, papaya, grapefruit, natsudaidai, tangerine, clementine, mandarin orange, currant, watermelon, honeydew melon, cantaloupe, lemon, lime, pear, blueberry, blackberry, raspberry, or strawberry juice, or a vegetable juice such as tomato, carrot, celery, cucumber, spinach, lettuce, watercress, sprouts, beet, herbs, cabbage, or wheat grass juice, or mixtures of juices), a juice concentrate, coffee, tea, a smoothie, a shake, soy milk, rice milk, a frappe, a milk fluid (e.g., full fat milk, 1% milk, 2% milk, heavy cream, half and half, whipping cream, or light cream), a meal replacement beverage, a diet beverage, or a nutritional supplement beverage. A particulate plant sterol composition can be incorporated in a food composition, e.g., a flour (e.g., a white, wheat, rye, soy, or rice flour), a baked good (e.g., a bread, a donut, a bagel, a muffin, a scone), candy, ice cream, a confection, an egg liquid, a liquid egg replacement, ice cream, yogurt, a health supplement, a meal replacement food, or a nutritional supplement. In certain solid food compositions, a particulate plant sterol composition can be first dispersed in a liquid, such as an aqueous composition, and then incorporated into the solid food composition (e.g., breads). Alternatively, the plant sterol composition can be mixed with other solid and/or powdered food compositions (e.g., flours).

Processes for Preparing Particulate Plant Sterol Compositions

Particulate plant sterol compositions described previously can be prepared using impact-and/or attrition-type milling techniques orjet- or vortex-milling techniques. The described techniques can be performed in a single pass, i.e. without the need for size classification of the mill discharge and recycling of improperly sized material to the mill.

In one process, a plant sterol starting material, as described above, is pre-cooled and then subjected to impact- or attrition-milling. For example, a plant sterol starting material can be cooled in the range from about −100° F. to about −275° F., or from about −175 to about −250° F. In certain embodiments, the plant sterol starting material is cooled to about −225° F. The plant sterol starting material can be cooled with liquid nitrogen, thereby resulting in an inert (N₂ gas) atmosphere for milling. By pre-cooling the plant sterol starting material, it is believed that the plant sterols become friable and pulverize easily. A pre-cooled plant sterol starting material can be subjected to impact- or attrition-milling in the presence of one or more of the following: a flow agent, a colorant, a flavorant, a vitamin, a mineral, a source of fiber, a protein, or a nutritional additive, as described previously. A pre-cooled plant sterol starting material can be subjected to impact- or attrition-milling in an inert atmosphere, e.g., N₂ gas.

Impact- or attrition-milling can be performed with a gap mill. Gap mills typically include a plurality of flat blades arranged around a conical-shaped rotor and a corresponding conical ribbed stator. Size reduction is accomplished in part by the impact of particles with the rotor and stator, but predominantly by particle-particle collisions. Typically, a rotor-stator gap is in the range of from about 0.025″ to about 0.05″. For example, in certain embodiments, the rotor-stator gap is about 0.03″. The rotor speed is adjustable so that an average tip speed of from about 110 m/s to about 150 m/s is achieved. In certain embodiments, an average tip speed is from about 120 to about 135 m/s. Gap mills are available commercially from MicrotecMicrotec (e.g., Microtec-Microtec Gap Mill), Bauermeister (e.g., Bauermeister ASIMA mill), Netzsch, and Hosokawa-Bepex.

Impact- and/or attrition-type milling techniques do not require the product to pass through a sizing screen. In certain embodiments, the impact- or attrition-milling, e.g., as performed with a gap mill, is performed in a single pass. After particle size reduction, a particulate plant sterol composition can be discharged, e.g., from a gap mill, at a temperature from about −25 to about −275° F. (e.g., at a temperature from about −40 to about −75° F., or at a temperature from about −40 to about −50° F.). In some embodiments, the composition is discharged at about −75° F.

In another process for preparing a particulate plant sterol composition, a plant sterol starting material is milled in ajet- or vortex-mill. In these mills, the driving force for comminution is derived from the high volumetric flows of high pressure air or other gas, such as N₂, which will thereby produce an inert atmosphere for milling. Comminution is mainly by particle-particle collisions, and the heat generated in the process is absorbed by the gas cooling upon expansion from, for example, 5 to 6 bar to atmospheric pressure and dissipated by the high gas flow. Particle-particle forces result in fine comminution of the starting material, which does not exit until it has achieved a minimal particle size, according to the design of the chamber and the vortex. Vortex mills are available from SuperFine Ltd., INOX Ltd., and Netzsch, or as described in U.S. Pat. No. 5,855,326.

In the present process, a vortex- or jet-mill can have an inlet air pressure of from about 5 to about 6 bar. Milling can be performed at ambient temperature, e.g., about 60 to about 80° F. A vortex-mill or jet-mill can have an outlet temperature of less than about 100° F. As indicated previously, milling with a vortex- or jet-mill can be performed in a single pass, and in the presence of one or more of the following: a flow agent, a colorant, a flavorant, a vitamin, a mineral, a source of fiber, a protein, or a nutritional additive, as described previously. Vortex- or jet-milling can be performed in an inert atmosphere.

While not being bound by theory, it is believed that the impact/attrition or vortex milling processes described herein do less oxidative degradation damage to the plant sterols in the plant sterol starting material than other methods that employ a melt of the plant sterol starting material and/or allow the starting material to contact air at temperatures from about 300 to 400° F.

The invention also provides a method for preparing an aqueous dispersion of a particulate plant sterol composition. In the method, a particulate plant sterol composition is mixed with an aqueous material. The particulate plant sterol composition demonstrates defined multi-peak volume/mass-weighted and surface area-weighted PSDs, as described previously. In certain cases, the dispersion can be heated gently (e.g., to 90 to 212 ° F.) for a brief period of time (e.g., 1 sec to 1 min), and/or homogenized. See, e.g., U.S. patent publication No. 2003/0232118A1.

In another embodiment, the invention provides a method for preparing a dispersion of a particulate plant sterol composition. In the method, a particulate plant sterol composition is homogenized with a pulp. A pulp can be any type of pulp, including, without limitation, fruit and vegetable pulps, such as citrus pulp (e.g., orange, lime, lemon, and grapefruit pulp); apple pulp; pear pulp; plum pulp, peach pulp; cherry pulp, mango pulp; guava pulp; papaya pulp; and assorted berry pulps. A pulp can contain about 2-8% pectin, or any value therebetween (e.g., about 2, 3, 4, 5, 6, 7, or 8%). In certain cases, a pulp containing about 5% pectin can be used. Water may be included in the homogenization process to help fluidize the pulp. If water is used, the ratio of water to pulp can be about 1:1 to about 4:1, or any value therebetween (e.g., about 1.5:1, 2:1, 2.5:1, 3:1, or 3.5:1). In certain cases, a ratio of 3:1 of water to pulp is used. Particulate plant sterols can be included at about 1% to about 10% by total weight of a water/pulp/particulate plant sterol mixture prior to homogenization, or any value therebetween (e.g., about 1.2, 1.5, 1.8, 2, 2.2, 2.5, 2.8, 3, 3.2, 3.5, 3.8, 4, 4.2, 4.5, 4.8, 5, 5.2, 5.5, 5.8, 6, 6.2., 6.5, 6.8, 7, 7.2, 7.5, 7.8, 8, 8.2, 8.5, 8.8, 9, 9.2, 9.5, or 9.8% by weight). In certain cases, particulate plant sterol compositions can be included at about 2-3% by weight.

In the method, a pulp, water, and a particulate plant sterol composition can be mixed prior to homogenization. For example, a pulp, water, and a particulate plant sterol composition can be pre-mixed with high shear (e.g., about 10,000 rpm) using a bench top mixer. Premixing can occur until the particulate plant sterol compositions are well dispersed in the pulp/water mixture, e.g., about 1 to 10 minutes, or about 5 minutes. The pulp/water/particulate plant sterol mixture can then be homogenized. The pulp/water/particulate plant sterol mixture can be homogenized in more than one stage. For example, the pulp/water/particulate plant sterol mixture can be homogenized in two stages. In certain cases, the pulp/water/particulate plant sterol mixture is homogenized at about 3000-5000 psi in a first stage, and then homogenized at about 300-800 psi in a second stage. In some cases, the pulp/water/particulate plant sterol mixture is homogenized at 4500 psi in a first stage and 500 psi in a second stage. Multiple passes can also be used. A homogenized pulp/water/particulate plant sterol mixture can deliver about 0.5 to about 1.5 g, or any value therebetween (e.g., about 0.6, 0.7, 0.8, 0.9, 1.0, 1.1, 1.2, 1.3, or 1.4 g) of particulate plant sterols per 10 g of pure pulp (e.g., without added water, wet basis).

After homogenization, a homogenized pulp/water/particulate plant sterol composition mixture can be incorporated in a food or beverage composition, as described previously. In other cases, a homogenized pulp/water/particulate plant sterol mixture can be added to an aqueous medium (as described previously), and mixed to result in an aqueous dispersion of particulate plant sterols. In certain cases, an aqueous medium can be a juice, such as a single strength juice such as orange juice or cranberry juice. In other cases, an aqueous medium can include water, a homogenized pulp/water/particulate plant sterol mixture, and a fruit or vegetable juice concentrate. An aqueous dispersion can contain about 0.3 g to about 1.8 g (or any value therebetween, e.g., about 0.4, 0.5, 0.6, 0.7, 0.8, 0.9, 1.0, 1.1, 1.2, 1.3, 1.4, 1.5, 1.6, or 1.7) of particulate plant sterols per serving (typically, 6-12 oz.).

While not being bound by any theory, it is believed that pulp, or some component of pulp such as pectins or terpenes, effectively homogenizes particulate plant sterol compositions, resulting in good dispersion, viscosity, and stabilization of the homogenized particulate plant sterol compositions. In addition, when incorporated in an aqueous medium, the resulting compositions have favorable mouthfeel and sensory characteristics (e.g., they are not chalky, oily, gritty, or astringent; and demonstrate no or minimal ringing, separation, or appearance of white flecked particulates, known as “floaters”).

### EXAMPLES

### Example 1

### Preparation of Spray-Prilled Particulate Plant Sterols (SP-1)

5 kg of 2 mm diameter sterol pastilles (Cargill, Incorporated, 90% by weight a mixture of β-sitosterol, campesterol, stigmasterol, brassicasterol, campestanol, β-sitostanol, and Δ-5-avenosterol) were melted in a Parr reactor and forced under a N_{2(g)} pressure of 35 psig into a Spraying Systems, Inc. SU-42 two-fluid nozzle at a rate of 2.7 gph, with the second, atomizing fluid being 3.6 scfm of 90 psig air; the fluid temperatures were 375° F. The product was collected in a conical chamber under atmospheric pressure and ambient temperature. Particle size analysis on a Horiba LA-910 Particle Size Analyzer [See Table I] showed the sterols to have 6 volume-% (as determined on a volume-weighted PSD) and 56 surface-area %- (as determined on a surface-area weighted PSD) in the “fines” region (particles less than 2 microns in diameter), with the “fines” peak mean diameter being 0.6 microns or greater. The calculated specific area of the overall material was 1.1 m²/g.

The product from Example 1 was incorporated at a level of 4.25 g/L into orange juice (see Example 9). Sensory evaluation of the juice proved it to be unacceptably “powdery/chalky” in mouthfeel, giving a drying sensation in the mouth.

### Example 2

### Preparation of Cryo-Milled Particulate Plant Sterols (CG-79)

Sterol pastilles (as described above) 2 mm in diameter were cooled in-line to an inlet temperature of about −175° F. before entering a Microtec Model 200 “Gap” Mill with a 0.030″ gap. The pre-cooled sterol pastilles were milled in a single pass at a feed rate of about 465 #/hr and a rotor speed of 12,000 rpm. The mill discharge temperature was −45 to −50° F. The product was analyzed on a Horiba LA-910 Particle Size Analyzer. See Table I.

CG-79 was evaluated in the orange juice test described in Examples 1 and 9. The product was evaluated as not acceptable.

### Example 3

### Preparation of Crvo-Milled Particulate Plant Sterols (CG-56)

Sterol pastilles (as described above) 2 mm in diameter were cooled in-line to an inlet temperature of about −245° F. before entering a Microtec Model 200 “Gap” Mill with a 0.030″ gap. The pre-cooled sterol pastilles were milled in a single pass at a feed rate of about 630 #/hr and a rotor speed of 12,000 rpm. The mill discharge temperature was −75° F. The product was analyzed on a Horiba LA-910 Particle Size Analyzer (see Example 8). See Table I.

### Example 4

### Preparation of Crvo-Milled Particulate Plant Sterols (CG-522)

Sterol pastilles (as described above) 2 mm in diameter were cooled in-line to an inlet temperature of about −225° F. before entering a Microtec Model 200 “Gap” Mill with a 0.030″ gap. The pre-cooled sterol pastilles were milled in a single pass at a feed rate of about 500-550 #/hr and a rotor speed of 12,000 rpm. Single pass milling was performed for about 3.5 hr total (1700# of sterol pastilles). The product was analyzed on a Horiba LA-910 Particle Size Analyzer. The mill discharge temperature was −75° F. See Table I.

### Example 5

### Preparation of Votex-Milled Particulate Plant Sterols (SF-1)

Sterol pastilles (as described above) 2 mm in diameter were drawn at a rate of about 4 kg/hr into a 6″D SuperFine, Ltd. Vortex Mill with no cooling of the material. The driving force for the mill was an inlet stream of air at a pressure of about 5.5 bar. The product was analyzed on a Horiba LA-910 Particle Size Analyzer. See Table I.

The product from Example 5 was incorporated into orange juice, as described in Examples 1 and 9. The material was rated excellent in the test juice application, with no detectable “powdery/chalky” mouthfeel or mouth-drying effect.

### Example 6

### Characterization of Spray-Prilled, Crvo-Milled, and Vortex-Milled Particulate Plant Sterols

Samples SP-1, CG-79, CG-56, CG-522, and SF-1 were analyzed on a Horiba LA-910 Particle Size Analyzer and the distribution of particles plotted as either volume (or mass)-weighted PSD vs. particle diameter or surface area-weighted PSD vs. particle diameter. Each composition's total specific surface area was also calculated from the particle size distribution. Results are set forth in Table I below.

TABLE I

| | SP-1 | CG-79 | CG-56 | CG-522 | SF-1 |
|---|---|---|---|---|---|
| “Fines” Peak only, | 6 | 13.1 | 18.3 | 19 | 30 |
| Volume-Weighted PSD- | | | | | |
| % of Total | | | | | |
| “Fines” Peak only, | 56 | 75 | 79 | 83 | 85 |
| Surface Area-Weighted | | | | | |
| PSD - % of Total | | | | | |
| “Fines” Peak only, | 0.6 | 0.6 | 0.5 | 0.4 | 0.4 |
| Surface Area-Weighted | | | | | |
| PSD - Mean Diameter [μ] | | | | | |
| Specific Surface Area | 1.1 | 2.0 | 2.9 | 4.4 | 6.1 |
| T tal Distribution - | | | | | |
| calculated from PSD | | | | | |
| [m²/g] | | | | | |
| Volume-Weighted PSD - | 3.7 | 9.4 | 0 | 0.3 | 0 |
| % of Material > 35 μ in | | | | | |
| diameter | | | | | |

### Example 7

### Sensory Evaluation of Particulate Plant Sterols in Orange Juice

The mouthfeel, flavor, and color/appearance of orange juice containing SP-1, CG-56, and SF-1 particulate plant sterol compositions at a concentration of 1 g/8 oz (240 mL) (see Example 9) were evaluated as blind, coded samples by a panel of 9 sensory judges in a round table discussion. None of the panelists had been trained specifically for this evaluation. The panelists were asked to describe the sensory characteristics of each sample, with special emphasis on mouth feel. SF-1 was determined to be the least chalky and most acceptable in terms of mouthfeel. The results are summarized in Table II.

TABLE II

| | | | | Accept- |
|---|---|---|---|---|
| | | | | ability |
| Sample | Color/Appearance | Flavor | Mouthfeel | Rank |
| 1. Un- | Orange-yellow | Typical of OJ, | Typical, no | 1 |
| treated | color, typical of | slightly | chalky feeling; | |
| Control | OJ | astringent | acceptable | |
| 2. CG- | Light yellow | Slightly watery, | Faint chalky | 3 |
| 56 | color, like a | less | mouthfeel, | |
| | smoothie; slight | characteristic | acceptable | |
| | oily film on top | OJ flavor | | |
| 3. SF-1 | Light yellow, like | More OJ flavor | Smooth, | 2 |
| | a smoothie, slight | than #2 | creamy | |
| | oily film on | | mouthfeel, | |
| | surface | | slightly more | |
| | | | viscous than | |
| | | | others; no | |
| | | | chalky | |
| | | | mouthfeel; | |
| | | | acceptable | |
| 4. SP-1 | Orange-yellow | Slightly watery, | Slightly chalky | 4 |
| | color, typical of | slightly | and powdery, | |
| | OJ; no oily film | astringent | unacceptable | |
| | on surface | | | |

### Example 8

### Particle Size Analysis

Particle size analysis of particulate plant sterol compositions was performed on a Horiba LA-910 Particle Size Analyzer as follows. Powdered particulate plant sterol samples were shaken in a bag to mix thoroughly. A balance was tared with a 15 mL conical tube on it, and 0.05-0.1 grams of sample was added to the 15 mL conical tube. Water was added to the 5 mL mark of the 15 mL conical tube. 6 drops of Triton X-100 (EM Science, CAS 9002-93-1) was added to the 15 mL conical tube using a disposable transfer pipet, and the conical tube was placed in an ultrasonic bath. After 1 minute of sonication, the mixture was stirred with a spatula, and resonicated for another 4 minutes. During the 4 minute sonication, the 15 ml conical tube was shaken 3 times. The contents of the 15 mL conical tube were transferred to a 7 mL tissue grinder. The sample was plunged 3 times using the pestle. Using a disposable 5¾″ borosilicate glass Pasteur pipet, inserted halfway into the liquid in the tissue grinder, ½ of a Pasteur pipet full of liquid was removed. All of the liquid in the Pasteur pipet was then dispensed into the instrument sample chamber containing 300 mL of DI water. After adding the sample solution to the instrument sample chamber, the sample chamber was sonicated for 1 minute, and the particle size distribution data acquired.

Analysis of Standard

A prepackaged standard solution (Duke Scientific Corp. 0.5 μm Particle Counter Size Standards or Duke Scientific Corp. 5.0 μm Particle Counter Size Standards) was added to the instrument sample chamber containing 100 mL of DI water until the % Transmittance was below 95%. The sample chamber was sonicated for 1 minute. The particle size distribution data was then acquired.

### Example 9

### Preparation of Homogenized Pulp and Particulate Plant Sterol Compositions and Dispersion into Test Juices

The following process was used to produce orange juice (from concentrate) containing 1 g particulate plant sterols per 240 mL:

Pulp Preparation

| | % by weight |
|---|---|
| Orange pulp | 24.30 |
| Water | 72.89 |
| Particulate plant sterols (e.g., CG-522, SF-1) | 2.81 |
| Total | 100.00 |

1. The pulp, water, and sterol mixture was premixed with high shear (10,000 rpm) for 5 minutes using a bench top high shear mixer (PowerGen 1800D, Fisher Scientific).

2. The premixed pulp, water, and sterol mixture was then homogenized (bench top homogenizer Model 15, APV Gaulin, Inc.) in two stages at 4500/500 psi.

Preparation of Single Strength Orange Juice

| | % by weight |
|---|---|
| Water | 65.41 |
| Sterol Containing Pulp Preparation | 16.46 |
| Frozen Concentrated Orange Juice | 18.13 |
| Total | 100.00 |

The ingredients listed above were blended with simple mixing.

This formula delivers about 1 g of particulate plant sterols via 10 g pulp (pure pulp (no added water) on a wet weight basis).

A number of embodiments of the invention have been described. Nevertheless, it will be understood that various modifications may be made without departing from the spirit and scope of the invention. Accordingly, other embodiments are within the scope of the following claims.

1. A composition comprising one or more particulate plant sterols, wherein said composition demonstrates a multi-peak volume-weighted or mass-weighted particle size distribution (PSD) of said one or more particulate plant sterols;
wherein said composition demonstrates a multi-peak surface-area-weighted PSD of said one or more particulate plant sterols; and
wherein said composition, when dispersed in a test juice, has an acceptable mouthfeel in said test juice.

2. The composition of claim 1, wherein said multi-peak volume- or mass-weighted PSD demonstrates:
a) a first peak of particulate plant sterols having a diameter less than 2 microns and a volume-weighted mean particle diameter of about 0.3 to about 0.5 microns; and
b) a second peak of particulate plant sterols having a diameter in the range from 2 to about 35 microns, wherein said second peak has a volume-weighted mean particle diameter of about 8 to about 12 microns.

3. The composition of claim 2, wherein said second peak represents from about 65% to about 85% of said volume- or mass-weighted PSD, and wherein said first peak represents from about 15% to about 35% of said volume- or mass-weighted PSD.

4. The composition of claim 1, wherein said composition demonstrates a multi-peak surface area-weighted PSD of said one or more particulate plant sterols.

5. The composition of claim 4, wherein said surface-area weighted PSD demonstrates:
a) a first peak of particulate plant sterols having a diameter less than 2 microns; and
b) a second peak of particulate plant sterols having a diameter in the range from 2 to about 35 microns, and wherein said second peak has a surface-area-weighted mean particle diameter of about 8 to 12 microns.

6. The composition of claim 5, wherein said first peak of particulate plant sterols represents from about 78% to about 92% of said surface-area weighted PSD.

7. The composition of claim 5, wherein said first peak of particulate plant sterols having a diameter less than 2 microns has a surface-area weighted mean particle diameter of about 0.5 microns or less.

8. The composition of claim 7, wherein said first peak of particulate plant sterols having a diameter less than 2 microns has a surface-area weighted mean particle diameter of from about 0.3 microns to about 0.5 microns.

9. The composition of claim 8, said first peak of particulate plant sterols having a diameter less than 2 microns has a surface-area weighted mean particle diameter of about 0.4 microns.

10. The composition of claim 4, wherein the total specific surface area of said multi-peak surface area-weighted PSD is greater than about 2 m²/g.

11. The composition of claim 10, wherein the total specific surface area of said multi-peak surface area-weighted PSD ranges from about 2.5 to about 7 m²/g.

12. The composition of claim 11, wherein the total specific surface area of said multi-peak surface area-weighted PSD ranges from about 2.8 to about 6.5 m²/g.

13. The composition of claim 3, wherein the volume-percentage of all particulate plant sterols having a diameter greater than 35 microns in said volume- or mass-weighted PSD is less than about 3%.

14. The composition of claim 13, wherein the volume-percentage of all particulate plant sterols having a diameter greater than 35 microns in said volume- or mass-weighted PSD is less than about 0.5%.

15. The composition of claim 1, wherein said composition is dispersible in an aqueous medium.

16. The composition of claim 1, wherein said composition is an aqueous composition.

17. The composition of claim 1, wherein said composition is a powdered composition.

18. The composition of claim 1, wherein said composition is a food or beverage composition.

19. The composition of claim 18, wherein said beverage composition is selected from the group consisting of a juice, a juice concentrate, coffee, tea, a smoothie, a shake, soy milk, rice milk, a frappe, a milk fluid, a meal replacement beverage, a diet beverage, and a nutritional supplement beverage.

20. The composition of claim 18, wherein said food composition is selected from the group consisting of a bread, a baked good, candy, ice cream, a confection, an egg, an egg replacement, ice cream, yogurt, a health supplement, a meal replacement food, and a nutritional supplement.

21. A process for preparing a particulate plant sterol composition comprising:
a) cooling a plant sterol starting material; and
b) subjecting said cooled plant sterol starting material to impact or attrition milling.

22. The process of claim 21, wherein said plant sterol starting material consists essentially of 1 or more plant sterols.

23. The process of claim 21, wherein said plant sterol starting material does not include an emulsifier.

24. The process of claim 21, wherein about 88% to about 100% by weight of said plant sterol starting material is 1 or more plant sterols.

25. The process of claim 24, wherein said plant sterol starting material includes vitamin E and/or tocopherol.

26. The process of claim 21, wherein said plant sterol starting material is cooled in the range from about −100° F. to about −275° F.

27. The process of claim 26, wherein said plant sterol starting material is cooled in the range from about −175 to about −250° F.

28. The process of claim 27, wherein said plant sterol starting material is cooled to about −225° F.

29. The process of claim 21, wherein said plant sterol starting material is cooled with liquid nitrogen.

30. The process of claim 21, wherein said impact or attrition milling is performed with a gap mill.

31. The process of claim 30, wherein said gap mill has a rotor-stator gap in the range of from about 0.025″ to about 0.05″.

32. The process of claim 31, wherein said rotor-stator gap is about 0.03″.

33. The process of claim 30, wherein said impact or attrition milling with said gap mill is performed in a single pass.

34. The process of claim 30, wherein said gap mill has an average tip speed of from about 110 m/s to about 150 m/s.

35. The process of claim 34, wherein said average tip speed is from about 120 to about 135 m/s.

36. The process of claim 30, wherein said particulate plant sterol composition is discharged from said gap mill at a temperature from about −25 to about −275° F.

37. The process of claim 36, wherein said particulate plant sterol composition is discharged from said gap mill at a temperature from about −40 to about −75° F.

38. The process of claim 37, wherein particulate plant sterol composition is discharged from said gap mill at a temperature from about −40 to about −50° F.

39. The process of claim 21, wherein said plant sterol starting material is in the form of pastilles having a diameter of from about 1 mm to about 4 mm.

40. The process of claim 39, wherein said pastilles have a diameter of about 2 mm.

41. The process of claim 21, wherein said cooled plant sterol starting material is subjected to impact or attrition milling in the presence of one or more of the following: a flow agent, a colorant, a flavorant, a vitamin, a mineral, a source of fiber, a protein, and a nutritional additive.

42. The process of claim 41, wherein said flow agent is sodium aluminosilicate.

43. The process of claim 41, wherein said colorant is β-carotene.

44. The process of claim 41, wherein said mineral is calcium.

45. A process for preparing a particulate plant sterol composition comprising:
a) milling a plant sterol starting material in a vortex mill having an inlet air pressure of from about 5 to about 6 bar and an outlet temperature of less than about 100° F.

46. The process of claim 45, wherein said milling is performed at a temperature from about 60 to about 80° F.

47. The process of claim 45, wherein said milling in said vortex mill is performed in a single pass.

48. The process of claim 45, wherein said plant sterol starting material consists essentially of 1 or more plant sterols.

49. The process of claim 45, wherein said plant sterol starting material does not include an emulsifier.

50. The process of claim 45, wherein about 88%-100% by weight of said plant sterol starting material is 1 or more plant sterols.

51. The process of claim 50, wherein said plant sterol starting material includes vitamin E and/or tocopherol.

52. The process of claim 45, wherein said plant sterol starting material is milled in the presence of one or more of the following: a flow agent, a colorant, a flavorant, a vitamin, a mineral, a source of fiber, a protein, and a nutritional additive.

53. The process of claim 52, wherein said flow agent is sodium aluminosilicate.

54. The process of claim 52, wherein said colorant is β-carotene.

55. The process of claim 52, wherein said mineral is calcium.

56. The process of claim 45, wherein said plant sterol starting material is in the form of pastilles having a diameter of from about 1 mm to about 4 mm.

57. The process of claim 56, wherein said pastilles have a diameter of about 2 mm.

58. A particulate plant sterol composition prepared by the process of claim 21.

59. A particulate plant sterol composition prepared by the process of claim 45.

60. A method for preparing an aqueous dispersion of a particulate plant sterol composition, comprising mixing a particulate plant sterol composition with an aqueous material, wherein said particulate plant sterol composition demonstrates a multi-peak surface area-weighted PSD.

61. The method of claim 60, wherein said multi-peak surface-area weighted PSD demonstrates:
a) a first peak of particulate plant sterols having a diameter less than 2 microns; and
b) a second peak of particulate plant sterols having a diameter of 2 to about 35 microns, wherein said second peak has a surface-area weighted mean particle diameter in the range from about 8 to about 12 microns.

62. The method of claim 61, wherein said first peak of particulate plant sterols represents from about 78% to about 92% of said surface-area weighted PSD.

63. The method of claim 61, wherein said first peak of particulate plant sterols having a diameter less than 2 microns has a surface-area weighted mean particle diameter of about 0.5 microns.

64. The method of claim 61, wherein said first peak of particulate plant sterols having a diameter less than 2 microns has a surface-area weighted mean particle diameter of from about 0.3 microns to about 0.5 microns.

65. The method of claim 64, wherein said first peak of particulate plant sterols having a diameter less than 2 microns has a surface-area weighted mean particle diameter of about 0.4 microns.

66. The method of claim 61, wherein the total specific surface area of said multi-peak surface area-weighted PSD is greater than about 2 m²/g.

67. The method of claim 66, wherein the total specific surface area of said multi-peak surface area-weighted PSD ranges from about 2.5 to about 7 m²/g.

68. The method of claim 67, wherein the total specific surface area of said multi-peak surface area-weighted PSD ranges from about 2.8 to about 6.5 m²/g.

69. The method of claim 61, wherein said aqueous material is selected from water, juice, a juice concentrate, coffee, tea, an egg mixture, ice cream, yogurt, soy milk, and a milk-based fluid.

70. A composition comprising a dispersion of a particulate plant sterol composition in an aqueous material, wherein said particulate plant sterol composition demonstrates a multi-peak surface area-weighted PSD, and wherein said multi-peak surface-area weighted PSD demonstrates:
a) a first peak of particulate plant sterols having a diameter less than 2 microns; and
b) a second peak of particulate plant sterols having a diameter of 2 to about 35 microns, wherein said second peak has a surface-area weighted mean particle diameter of about 8 to about 12 microns.

71. The composition of claim 70, wherein said composition has no or only a slight detectable chalky mouthfeel.

72. The method of claim 60, wherein said particulate plant sterol composition is mixed with said aqueous material in order to substantially avoid an undesirable sensory attribute in said aqueous dispersion.

73. The method of claim 72, wherein said undesirable sensory attribute is a chalky, gritty, drying, or powdery mouthfeel.

74. A method for preparing a dispersion of a particulate plant sterol composition, comprising homogenizing a particulate plant sterol composition with a pulp.

75. The method of claim 74, wherein said homogenizing step further includes water.

76. The method of claim 75, wherein prior to said homogenizing, said pulp, said water, and said particulate plant sterol composition are mixed with high shear.

77. The method of claim 74, wherein said homogenizing step is performed in two stages.

78. The method of claim 77, wherein said first homogenizing stage is performed at a higher psi than said second homogenizing stage.

79. The method of claim 74, wherein said pulp is a citrus pulp selected from orange pulp, lemon pulp, lime pulp, and grapefruit pulp.

80. The method of claim 75, wherein said water:pulp ratio by weight is about 1:1 to about 4:1.

81. The method of claim 75, wherein said particulate plant sterol composition is about 1% to about 10% by weight of said total amount of said pulp, said water, and said particulate plant sterol composition.

82. A method for preparing an aqueous dispersion of a particulate plant sterol comprising mixing a homogenized mixture containing a pulp and a particulate plant sterol composition with an aqueous medium.

83. The method of claim 82, wherein said aqueous medium is a juice or juice concentrate.
